(19) United States
(12) Patent Application Publication
(10) Pub. No.: US 2023/0404995 A1
(43) Pub. Date: Dec. 21, 2023

(54) COMPOSITIONS AND METHODS FOR TREATING PULMONARY FIBROSIS

(71) Applicant: Pulmosim Therapeutics LLC, San Francisco, CA (US)

(72) Inventor: Vivek GUPTA, Floral Park, NY (US)

(73) Assignee: Pulmosim Therapeutics LLC, San Francisco, CA (US)

(21) Appl. No.: 18/037,405

(22) PCT Filed: Nov. 17, 2021
(86) PCT No.: PCT/US2021/059757

§ 371 Date: May 17, 2023

Related U.S. Application Data
(60) Provisional application No. 63/115,106, filed on Nov. 18, 2020.

(51) Int. Cl. A61K 31/473; A61K 31/573; A61K 31/4418; A61K 33/00; A61K 9/51; A61K 31/496; A61P 11/00

(52) U.S. Cl. CPC A61K 31/473; A61K 31/573; A61K 31/4418; A61K 33/00; A61K 9/5153; A61K 31/496; A61P 11/00

(57) ABSTRACT

The present disclosure relates generally to compositions and methods for preventing, ameliorating or treating pulmonary fibrosis and/or reducing the severity of one or more risk factors, signs, or symptoms associated with pulmonary fibrosis.

### CROSS-REFERENCE TO RELATED APPLICATIONS

This application claims the benefit of and priority to U.S. Provisional Appl. No. 63/115,106, filed Nov. 18, 2020, the disclosure of which is incorporated by reference herein in its entirety.

### TECHNICAL FIELD

The present technology relates generally to compositions and methods for ameliorating, treating or preventing pulmonary fibrosis and/or reducing the severity of one or more risk factors, signs, or symptoms associated with pulmonary fibrosis.

### BACKGROUND

The following description of the background of the present technology is provided simply as an aid in understanding the present technology and is not admitted to describe or constitute prior art to the present technology.

Idiopathic pulmonary fibrosis (IPF) is a progressive disease restricted to the lung and characterized by progressive interstitial fibrosis of lung parenchyma. Currently, more than 80,000 adults in the United States have IPF, and more than 30,000 new cases are diagnosed each year. The interstitial fibrosis associated with IPF leads to progressive loss of lung function, resulting in death due to respiratory failure in most patients. The median survival from the time of diagnosis is 2-3 years (Raghu et al., Am J Respir Crit Care Med 183:788-824 (2011)). The etiology and key molecular and pathophysiological drivers of IPF are unknown. The only treatment shown to prolong survival in IPF patients is lung transplantation (Thabut et al., Annals of internal medicine 151:767-774 (2009)). Lung transplantation, however, is associated with considerable morbidity, not all IPF patients are appropriate candidates for it, and there is a relative paucity of suitable donor lungs. Despite numerous attempts, no drug therapies to date have been shown to substantially prolong survival in a randomized, placebo-controlled interventional trial in IPF patients.

### SUMMARY OF THE PRESENT TECHNOLOGY

In one aspect, the present disclosure provides a method for treating or preventing pulmonary fibrosis in a subject in need thereof, comprising administering to the subject a therapeutically effective amount of quinacrine or a pharmaceutically acceptable salt thereof. In some embodiments, the subject has been diagnosed as having idiopathic pulmonary fibrosis.

In any and all embodiments of the methods disclosed herein, the signs or symptoms of pulmonary fibrosis comprise one or more of dry cough, chest pain, loss of appetite, shortness of breath, fatigue, weight loss, clubbing (a widening and rounding of the tips of the fingers), joint and muscle aches, swelling of the legs, epithelial to mesenchymal transition (EMT), myofibroblast activation, and severe progressive fibrosis including fibrotic foci and honeycombing. In some embodiments, EMT is characterized by loss of epithelial markers, cytoskeletal reorganization, and transition to a spindle-shaped morphology with acquisition of mesenchymal markers.

Additionally or alternatively, in some embodiments of the methods disclosed herein, the subject is human.

In any and all embodiments of the methods disclosed herein, the quinacrine or pharmaceutically acceptable salt thereof is administered orally, topically, intranasally, systemically, intravenously, subcutaneously, intraperitoneally, intradermally, intraocularly, iontophoretically, transmucosally, or intramuscularly.

Additionally or alternatively, in some embodiments, the quinacrine or pharmaceutically acceptable salt thereof is administered in an effective amount between about 1 mg/kg to about 15 mg/kg or between about 0.5 μM to about 10 μM. In certain embodiments, the quinacrine or pharmaceutically acceptable salt thereof is administered in an effective amount of about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, about 10 mg/kg, about 10.5 mg/kg, about 11 mg/kg, about 11.5 mg/kg, about 12 mg/kg, about 12.5 mg/kg, about 13 mg/kg, about 13.5 mg/kg, about 14 mg/kg, about 14.5 mg/kg, or about 15 mg/kg. In some embodiments, the quinacrine or pharmaceutically acceptable salt thereof is administered in an effective amount of about 0.5 μM, about 1 μM, about 1.5 μM, about 2 μM, about 2.5 μM, about 3 μM, about 3.5 μM, about 4 μM, about 4.5 μM, about 5 μM, about 5.5 μM, about 6 μM, about 6.5 μM, about 7 μM, about 7.5 μM, about 8 μM, about 8.5 μM, about 9 μM, about 9.5 μM, or about 10 μM.

Additionally or alternatively, in some embodiments, the methods of the present technology further comprise separately, sequentially or simultaneously administering one or more additional therapeutic agents to the subject. Examples of additional therapeutic agents include, but are not limited to, nintedanib, pirfenidone, corticosteroids, and oxygen therapy.

Additionally or alternatively, in some embodiments of the methods disclosed herein, the subject exhibits reduced hyper-proliferation of lung fibroblasts, and/or decreased lung scarring following administration of quinacrine or pharmaceutically acceptable salt thereof. Additionally or alternatively, in some embodiments, the subject exhibits a decrease in ECM production, reduced collagen expression, and/or an increase in E-cadherin following administration of quinacrine or pharmaceutically acceptable salt thereof. Additionally or alternatively, in some embodiments, the quinacrine or pharmaceutically acceptable salt thereof is administered daily for 1 week, 2 weeks, 3 weeks, 4 weeks, 6 weeks, 12 weeks or more.

Additionally or alternatively, in some embodiments of the methods disclosed herein, the quinacrine or pharmaceutically acceptable salt thereof is encapsulated in nanoparticles. In some embodiments, the nanoparticles are PLGA hybrid nanoparticles.

Additionally or alternatively, in some embodiments, the IPF is induced by TGF-β signaling, PDGF-BB signaling, FGF signaling, or exposure to bleomycin.

### BRIEF DESCRIPTION OF THE DRAWINGS

FIGS. 1A-D demonstrate anti-fibrotic efficacy of quinacrine (QA) in A549 lung epithelial cells. FIG. 1A shows phase-contrast images demonstrating QA's efficacy in inhibiting TGF-β induced epithelial-mesenchymal transition (EMT). FIG. 1B shows fluorescent images showing loss of E-cadherin following TGF-β treatment (5 ng/ml, 48 h), and its recovery with QA (2.5 μM) treatment. FIGS. 1C-D show western blot quantification of E-cadherin as compared to β-actin. All images are representative of n=3 experiments. Data represent mean±SD (n=3). *p<0.05. Scale bar=100 μm. FIG. 1E shows the chemical structure of quinacrine.

FIGS. 2A-2D demonstrate in-vitro efficacy of QA in inhibition of fibrotic responses induced by growth factors. FIG. 2A shows QA-mediated inhibition of TGF-β (10 ng/ml)-induced cellular hyper-proliferation of WI-38 lung fibroblasts. FIG. 2B shows QA-mediated inhibition of PDGF-BB (10 ng/ml)-induced cellular hyper-proliferation of WI-38 lung fibroblasts. FIG. 2C shows QA-mediated inhibition of FGF (10 ng/ml)-induced cellular hyper-proliferation of WI-38 lung fibroblasts. Data represent mean±SD (n=3). *p<0.05; ***p<0.001; and ****p<0.0001. FIG. 2D shows reduction in TGF-β (5 ng/ml) induced collagen production with QA (2.5 μM) treatment for 48 hours. All images are representative of n=3 experiments. Scale bar=100 μm.

FIG. 3 shows in vitro cytotoxicity of QA in primary human bronchial epithelial (HBE) cells. Cell viability studies were carried out on human bronchial epithelial (HBE) cells using Cell Titer Blue cell viability kit.

FIG. 4 shows that QA inhibits TGF-beta induced epithelial-mesenchymal transition (EMT) in HBE cells, one of the major hallmarks of IPF pathogenesis and progression.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

The present disclosure provides the methods of preventing, ameliorating or treating pulmonary fibrosis and/or reducing the severity of one or more risk factors, signs, or symptoms associated with pulmonary fibrosis in a subject in need thereof comprising administering to the subject an effective amount of quinacrine. As demonstrated in the Examples described herein, quinacrine (i) protects alveolar epithelial cells from TGF-beta induced fibrotic response, and (ii) inhibits TGF-beta induced epithelial-mesenchymal transition (EMT) in human bronchial epithelial cells. Accordingly, quinacrine is useful in methods for preventing, ameliorating or treating pulmonary fibrosis, in particular, IPF.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “gene” means a segment of DNA that contains all the information for the regulated biosynthesis of an RNA product, including promoters, exons, introns, and other untranslated regions that control expression.

As used herein, the terms “individual”, “patient”, or “subject” can be an individual organism, a vertebrate, a mammal, or a human. In some embodiments, the individual, patient or subject is a human.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20^{th} edition, ed. A. Gennaro, 2000, Lippincott, Williams & Wilkins, Philadelphia, Pa.).

As used herein, “prevention” or “preventing” of a disorder or condition refers to a compound that, in a statistical sample, reduces the occurrence of the disorder or condition in the treated sample relative to an untreated control sample, or delays the onset of one or more symptoms of the disorder or condition relative to the untreated control sample. As used herein, preventing pulmonary fibrosis includes preventing or delaying the initiation of pulmonary fibrosis. As used herein, prevention of pulmonary fibrosis also includes preventing a recurrence of one or more signs or symptoms of pulmonary fibrosis.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the term “therapeutic agent” is intended to mean a compound that, when present in an effective amount, produces a desired therapeutic effect on a subject in need thereof.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment or prevention of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

### Idiopathic Pulmonary Fibrosis (IPF)

Idiopathic pulmonary fibrosis (IPF) is a restrictive lung disease characterized by progressive interstitial fibrosis of lung parenchyma. This interstitial fibrosis associated with IPF leads to progressive loss of lung function, resulting in death due to respiratory failure in most patients. Although the prognosis for all IPF patients is dire, there is considerable heterogeneity in disease trajectory (Raghu et al., Am J Respir Crit Care Med 183:788-824 (2011)). Some patients exhibit a relatively indolent course, losing lung function at a relatively constant rate over as long as 10 years or more, while others experience a more rapid decline in lung function, succumbing to death within a year or two of diagnosis. In addition, some patients suffer from acute exacerbations of the disease, typically characterized by sudden dramatic decreases in lung function. Generally, these patients do not fully recover after the acute event and often die during or shortly after an exacerbation. This heterogeneity in disease trajectory suggests that different IPF patients may have different pathophysiological factors underlying their disease. Given the limited understanding of the underlying molecular pathology, the variability in disease trajectory, and time and expense of conducting survival studies in unselected populations of IPF patients, designing appropriately powered clinical studies to assess the potential of a candidate therapeutic to prolong survival in IPF is extremely challenging.

In the presence of a surgical lung biopsy showing the histological appearance of usual interstitial pneumonia (UIP), the definite diagnosis of IPF requires the following (American Thoracic Society. Idiopathic pulmonary fibrosis: diagnosis and treatment. International consensus statement. American Thoracic Society (ATS) and the European Respiratory Society (ERS). Am J Respir Crit Care Med 2000; 161:646-64): (1) the exclusion of other causes of interstitial lung diseases (ILD), (2) abnormal pulmonary function studies that include evidence of restriction of lung capacity and/or impaired gas exchange or decreased diffusing capacity for carbon monoxide (DLCO), (3) abnormalities on conventional chest radiograph or high-resolution computed tomography (HRCT) scans. The criteria for diagnosis of IPF in the absence of a surgical lung biopsy necessitate the correlation between all clinical and radiological features.

Signs and/or symptoms of IPF include, but are not limited to, dry cough, chest pain, loss of appetite, shortness of breath, fatigue, weight loss, clubbing (a widening and rounding of the tips of the fingers), joint and muscle aches, swelling of the legs, epithelial to mesenchymal transition (EMT), myofibroblast activation, and severe progressive fibrosis including fibrotic foci and honeycombing.

### Methods of Treating Pulmonary Fibrosis Using the Compositions of the Present Technology

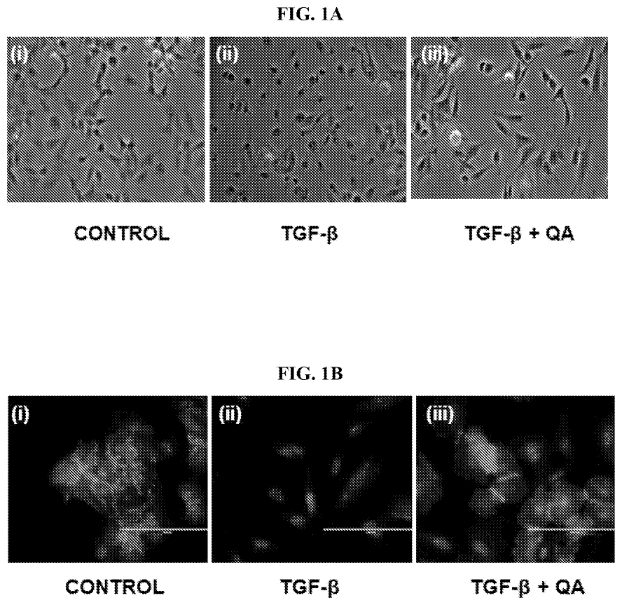

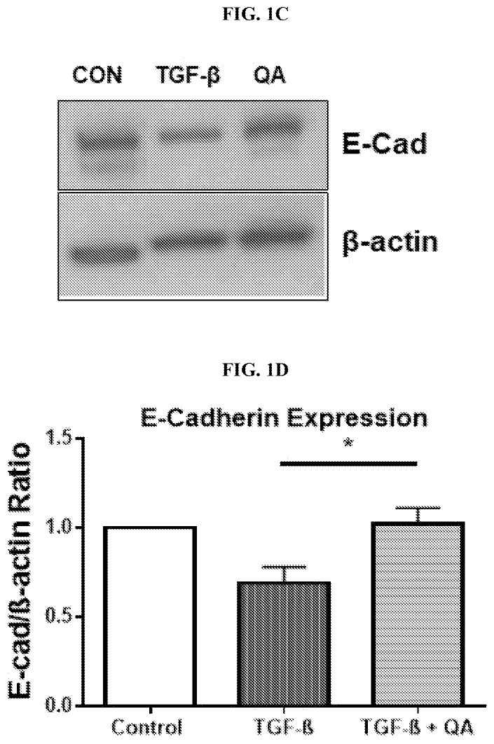

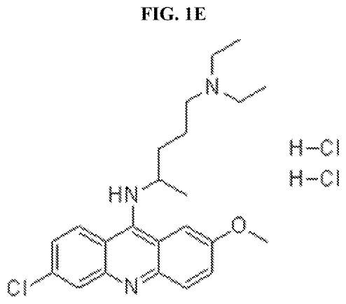

In one aspect, the present disclosure provides a method for treating or preventing pulmonary fibrosis in a subject in need thereof, comprising administering to the subject a therapeutically effective amount of the composition of the present technology (i.e., quinacrine or a pharmaceutically acceptable salt thereof). The chemical structure of quinacrine is shown in FIG. 1. In some embodiments, the subject has been diagnosed as having idiopathic pulmonary fibrosis (IPF). Additionally or alternatively, in some embodiments, the subject exhibits elevated expression of one or more genes selected from among CCL18 MUCL1, MUC4, MUC20, PRR7, PRR15, SPRR1B, SPRR2D, KRT5, KRT6B, KRT13, KRT14, KRT15, KRT17, SERPINB3, SERPINB4, SERPINB5, SERPINB13, CLCA2, TRPV4, BBS5, MMP3, SAA4, CXCR3, CXCR5, CXCL13, CCR6, CCR7, CD19, MS4A1 (CD20), TNFRSF17 (BCMA), BLK, BLNK, FCRLA, FCRL2, FCRL5, CD79A, CD79B, CD27, CD28, CD1A, CD1B, CD1C, CD1E, IGHV1-69, IGLJ3, IGJ, IGHV3-48, IGLV3-21, IGKV1-5, IGHG1, IGKC, IGLV6-57, IGK@(immunoglobulin kappa locus), IGHA1, IGKV2-24, IGKV1D-8, IGHM, COL14A1, COL15A1, COL1A1, COL1A2, COL5A2, COL12A1, COL18A1, COL16A1, CTHRC1, SCGF (CLEC11A), HGF, IGFBP7, LOXL1, LOXL2, GLI1, GLI2, SMO, SFRP2, DIO2, CDH11, POSTN, TGFB3, CHI3L1 (YKL-40), CCL13, COMP, CXCL13, MMP7, POSTN, SPP1 (OPN), CCL11, and CCL17.

In any and all embodiments of the methods disclosed herein, administration of quinacrine prevents, delays the onset of, or reduces the signs or symptoms of IPF. In some embodiments, the signs or symptoms of IPF comprise one or more of dry cough, chest pain, loss of appetite, shortness of breath, fatigue, weight loss, clubbing (a widening and rounding of the tips of the fingers), joint and muscle aches, swelling of the legs, epithelial to mesenchymal transition (EMT), myofibroblast activation, and severe progressive fibrosis including fibrotic foci and honeycombing.

Additionally or alternatively, in some embodiments, the subject exhibits reduced hyper-proliferation of lung fibroblasts, and/or decreased lung scarring following administration of quinacrine or pharmaceutically acceptable salt thereof.

Additionally or alternatively, in some embodiments, the methods of the present technology further comprise separately, sequentially or simultaneously administering one or more additional therapeutic agents to the subject. In some embodiments, the additional therapeutic agents are selected from the group consisting of: nintedanib, pirfenidone, corticosteroids, and oxygen therapy.

In any and all embodiments of the methods disclosed herein, the subject is a human.

### Modes of Administration and Effective Dosages

Any method known to those in the art for contacting a cell, organ or tissue with QA may be employed. Suitable methods include in vitro, ex vivo, or in vivo methods. In vivo methods typically include the administration of QA or pharmaceutically acceptable salt thereof to a mammal, suitably a human. When used in vivo for therapy, QA or pharmaceutically acceptable salt thereof is administered to the subject in effective amounts (i.e., amounts that have desired therapeutic effect). The dose and dosage regimen will depend upon the degree of the disease state of the subject, the characteristics of the particular composition used, e.g., its therapeutic index, the subject, and the subject's history.

The effective amount may be determined during pre-clinical trials and clinical trials by methods familiar to physicians and clinicians. An effective amount of QA or pharmaceutically acceptable salt thereof useful in the methods may be administered to a mammal in need thereof by any of a number of well-known methods for administering pharmaceutical compositions. The composition including QA or pharmaceutically acceptable salt thereof may be administered systemically or locally. In any and all embodiments of the methods disclosed herein, QA or pharmaceutically acceptable salt thereof is administered orally, topically, intranasally, via inhalation, intrapleurally, systemically, intravenously, subcutaneously, intraperitoneally, intradermally, intraocularly, iontophoretically, transmucosally, or intramuscularly.

QA may be formulated as a pharmaceutically acceptable salt. The term “pharmaceutically acceptable salt” means a salt prepared from a base or an acid which is acceptable for administration to a patient, such as a mammal (e.g., salts having acceptable mammalian safety for a given dosage regime). However, it is understood that the salts are not required to be pharmaceutically acceptable salts, such as salts of intermediate compositions that are not intended for administration to a patient. Pharmaceutically acceptable salts can be derived from pharmaceutically acceptable inorganic or organic bases and from pharmaceutically acceptable inorganic or organic acids. In addition, when a composition contains both a basic moiety, such as an amine, pyridine or imidazole, and an acidic moiety such as a carboxylic acid or tetrazole, zwitterions may be formed and are included within the term “salt” as used herein. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine and the like. Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucuronic, mandelic, mucic, nicotinic, orotic, pamoic, pantothenic, sulfonic acids (e.g., benzenesulfonic, camphosulfonic, edisylic, ethanesulfonic, isethionic, methanesulfonic, naphthalenesulfonic, naphthalene-1,5-disulfonic, naphthalene-2,6-disulfonic and p-toluenesulfonic acids), xinafoic acid, and the like.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration.

The composition described herein, or a pharmaceutically acceptable salt thereof, can be incorporated into pharmaceutical compositions for administration, singly or in combination, to a subject for the treatment or prevention of IPF described herein. Such compositions typically include the active agent and a pharmaceutically acceptable carrier. As used herein, the term “pharmaceutically acceptable carrier” includes saline, solvents, dispersion media, coatings, antibacterial and antifungal agents, isotonic and absorption delaying agents, and the like, compatible with pharmaceutical administration. Supplementary active compositions can also be incorporated into the compositions.

Dosage, toxicity and therapeutic efficacy of any therapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit high therapeutic indices are advantageous. While compositions that exhibit toxic side effects may be used, care should be taken to design a delivery system that targets such compositions to the site of affected tissue in order to minimize potential damage to uninfected cells and, thereby, reduce side effects.

The data obtained from the cell culture assays and animal studies can be used in formulating a range of dosage for use in humans. The dosage of such compositions may be within a range of circulating concentrations that include the ED50 with little or no toxicity. The dosage may vary within this range depending upon the dosage form employed and the route of administration utilized. For any composition used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

Typically, an effective amount of the composition, sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example dosages can be 1 mg/kg body weight or 10 mg/kg body weight every day, every two days or every three days or within the range of 1-10 mg/kg every week, every two weeks or every three weeks. In one embodiment, a single dosage of the composition ranges from 0.001-10,000 micrograms per kg body weight. In one embodiment, composition concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In some embodiments, a therapeutically effective amount of a composition may be defined as a concentration of the composition at the target tissue of 10⁻¹² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a human.

For therapeutic and/or prophylactic applications, a composition comprising QA or pharmaceutically acceptable salt thereof, is administered to the subject. In some embodiments, the QA or pharmaceutically acceptable salt thereof is administered in an effective amount between about 1 mg/kg to about 15 mg/kg. In certain embodiments, the QA or pharmaceutically acceptable salt thereof is administered in an effective amount of about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, about 10 mg/kg, about 10.5 mg/kg, about 11 mg/kg, about 11.5 mg/kg, about 12 mg/kg, about 12.5 mg/kg, about 13 mg/kg, about 13.5 mg/kg, about 14 mg/kg, about 14.5 mg/kg, or about 15 mg/kg. Additionally or alternatively, in some embodiments, the QA or pharmaceutically acceptable salt thereof is administered in an effective amount between about 1 μM to about 10 μM. In certain embodiments, the QA or pharmaceutically acceptable salt thereof is administered in an effective amount of about 1 μM, about 1.5 μM, about 2 μM, about 2.5 μM, about 3 μM, about 3.5 μM, about 4 μM, about 4.5 μM, about 5 μM, about 5.5 μM, about 6 μM, about 6.5 μM, about 7 μM, about 7.5 μM, about 8 μM, about 8.5 μM, about 9 μM, about 9.5 μM, or about 10 μM. Values and ranges intermediate to the recited values are also contemplated as part of the present disclosure.

In some embodiments, the QA or pharmaceutically acceptable salt thereof is administered one, two, three, four, or five times per day. In some embodiments, the QA or pharmaceutically acceptable salt thereof is administered more than five times per day. Additionally or alternatively, in some embodiments, the QA or pharmaceutically acceptable salt thereof is administered every day, every other day, every third day, every fourth day, every fifth day, or every sixth day. In some embodiments, the QA or pharmaceutically acceptable salt thereof is administered weekly, bi-weekly, tri-weekly, or monthly. In some embodiments, the QA or pharmaceutically acceptable salt thereof is administered for a period of one, two, three, four, or five weeks. In some embodiments, the QA or pharmaceutically acceptable salt thereof is administered for six weeks or more. In some embodiments, the QA or pharmaceutically acceptable salt thereof is administered for twelve weeks or more. In some embodiments, the QA or pharmaceutically acceptable salt thereof is administered for a period of less than one year. In some embodiments, the QA or pharmaceutically acceptable salt thereof is administered for a period of more than one year. In some embodiments, the QA or pharmaceutically acceptable salt thereof is administered throughout the subject's life.

In some embodiments, the QA or pharmaceutically acceptable salt thereof is administered daily for 1 week or more. In some embodiments of the methods of the present technology, the QA or pharmaceutically acceptable salt thereof is administered daily for 2 weeks or more. In some embodiments of the methods of the present technology, the QA or pharmaceutically acceptable salt thereof is administered daily for 3 weeks or more. In some embodiments of the methods of the present technology, the QA or pharmaceutically acceptable salt thereof is administered daily for 4 weeks or more. In some embodiments of the methods of the present technology, the QA or pharmaceutically acceptable salt thereof is administered daily for 6 weeks or more. In some embodiments of the methods of the present technology, the QA or pharmaceutically acceptable salt thereof is administered daily for 12 weeks or more. In some embodiments, the QA or pharmaceutically acceptable salt thereof is administered daily throughout the subject's life. Values and ranges intermediate to the recited values are also contemplated as part of the present disclosure.

### EXAMPLES

The present technology is further illustrated by the following Examples, which should not be construed as limiting in any way.

### Example 1: QA Protects Alveolar Epithelial Cells (A549) from TGF-β Induced Fibrotic Response

Epithelial-to-mesenchymal transition (EMT) is one of the key features of pathogenesis of fibrotic diseases. EMT has been known to be induced by TGF-β dependent Smad signaling. To determine the efficacy of QA in attenuating EMT in epithelial cells, serum starved A549 alveolar epithelial cells were incubated with TGF-β (5 ng/ml) along with simultaneous treatment with QA (2.5 μM). The treated cells were tested for morphological changes and for presence of E-cadherin, an epithelial marker, which is lost during transition to mesenchymal stem cells. Morphological studies confirmed the efficacy of QA in protecting A549 cells against TGF-β induced EMT, by reversing fibroblastic morphology induced by TGF-β treatment (FIG. 1A). Confirmatory evidence came from quantifying E-cadherin expression in epithelial cells following the treatment. As shown in FIG. 1B, TGF-β treatment significantly reduced E-cadherin expression (red fluorescence) in the cells, which was restored following simultaneous incubation with QA (FIG. 1B (iii)). The immunofluorescent results were confirmed with western blot experiments. A 40% reduction in E-cadherin expression was observed with TGF-β treatment, whereas E-cadherin expression was completely restored in cells that were co-incubated with QA and TGF-β (FIGS. 1C & 1D). These results demonstrate definitive role of QA in providing protection against development of fibrotic cells following upregulation of pathogenic pathways.

These results demonstrate that compositions including quinacrine or pharmaceutically acceptable salts thereof are useful in methods for preventing, ameliorating or treating pulmonary fibrosis and/or reducing the severity of one or more risk factors, signs, or symptoms associated with pulmonary fibrosis.

### Example 2: QA Treatment Reduces Growth Factor Induced Activation and Proliferation of Lung Fibroblasts (WI-38), and Accumulation of Collagen

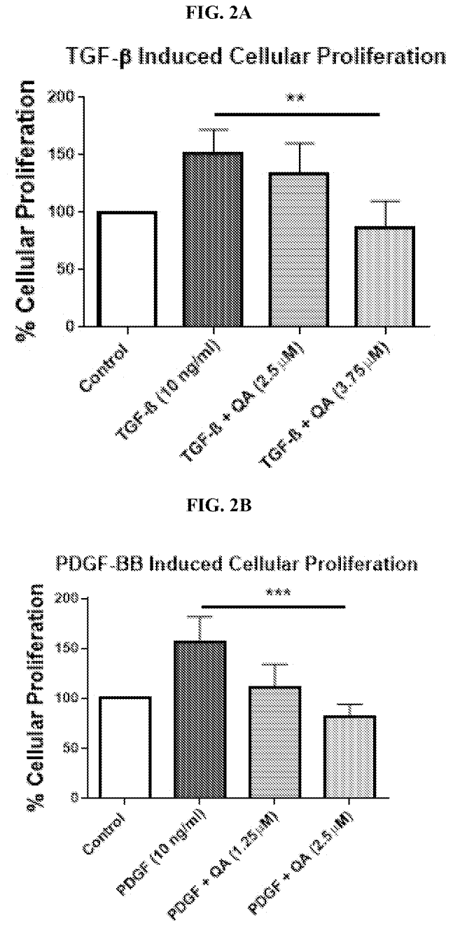

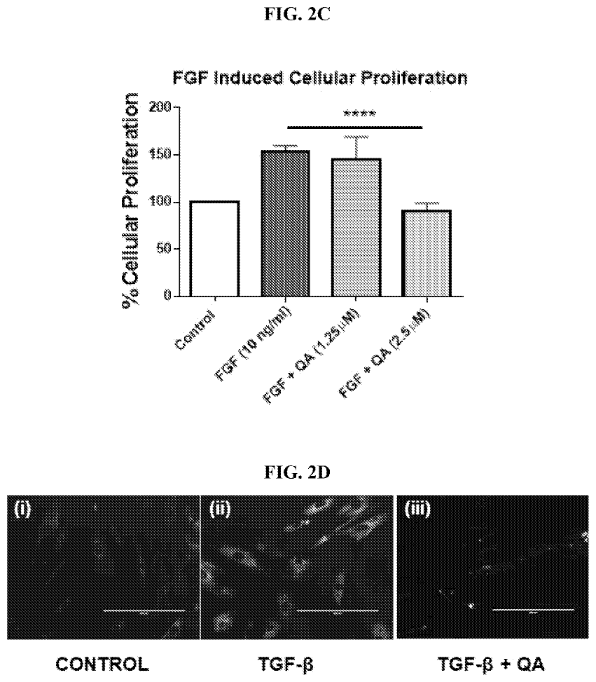

Following the anti-fibrotic response of QA in-vitro, the efficacy of QA in inhibiting hyper-proliferation of lung fibroblasts following induction with growth factors, TGF-β, PDGF-BB, and FGF was tested. To test QA's efficacy in inhibiting fibroblast hyper-proliferation and activation by measuring cell proliferation observed in response to induction with growth factors, TGF-β (10 ng/ml; 48 hours), and PDGF-BB/FGF (10 ng/ml; 72 hours). FIG. 2A shows dose-dependent reduction in fibroblast proliferation by QA in the presence of TGF-β, while FIGS. 2B & 2C show dose-dependent reduction in PDGF and FGF induced hyper-proliferation. Moreover, none of the doses of QA used in the study was cytotoxic (>90% viability), and the results can be truly attributed to QA's efficacy in inhibiting diseased cellular hyper-proliferation.

It is well established that during fibrotic diseases, fibroblasts and epithelial cells are transformed in myofibroblasts (mesenchymal cells), which secrete excess amount of extracellular matrix (ECM), one of the major components being interstitial collagen at the site of tissue injury. Serum starved WI-38 lung fibroblasts were activated with TGF-β (5 ng/ml; 48 hours), and QA's efficacy's in inhibiting collagen production due to transition of fibroblasts in collagen-secreting fibroblasts was tested. As shown in FIG. 2D (i)-(iii), TGF-β significantly enhanced collagen production in WI-38 cells (green fluorescence) as determined by immune-fluorescent imaging (FIG. 2D (ii)). However, collagen expression was significantly reduced by quinacrine (2.5 μM) (FIG. 2D (iii)), which underlines efficacy of QA in further inhibiting TGF-β induced fibrosis pathogenesis pathway. These results also outline the possible tyrosine kinase inhibitory activity of QA that is vital of IPF treatment due to known involvement of tyrosine kinases in IPF associated lung scarring and ECM production.

These results demonstrate that compositions including quinacrine or pharmaceutically acceptable salts thereof are useful in methods for preventing, ameliorating or treating pulmonary fibrosis and/or reducing the severity of one or more risk factors, signs, or symptoms associated with pulmonary fibrosis.

### Example 3: Cytotoxic Effects of QA in Primary Human Bronchial Epithelial (HBE) Cells

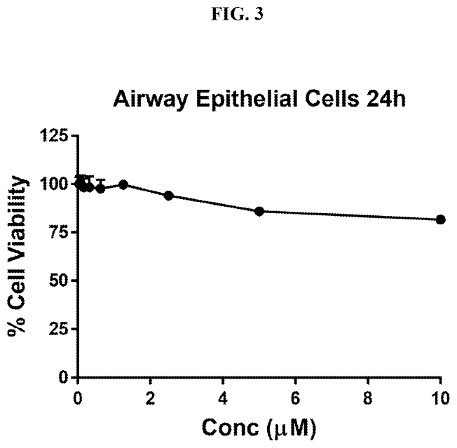

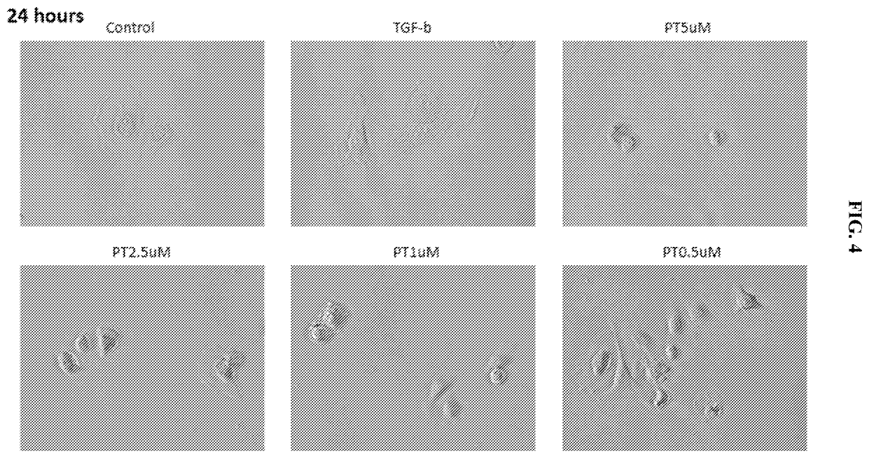

A cell viability study was carried out on HBE cells using Cell Titer Blue cell viability kit. Briefly, cells were treated with varying concentrations of QA (0.039-10 μM), for 24 hours, and media was used as a control. Fluorescence readings were taken at 540/590 nm using a Spark 10M Plate Reader (Tecan, Switzerland). As shown in FIG. 3, 24-hour QA treatment resulted in minimal toxicity to HBE cells, as determined by >85% cell viability.

These results demonstrate that compositions including quinacrine or pharmaceutically acceptable salts thereof are safe for use in methods for preventing, ameliorating or treating pulmonary fibrosis and/or reducing the severity of one or more risk factors, signs, or symptoms associated with pulmonary fibrosis.

### Example 4: QA Treatment Inhibits TGF-Beta Induced Epithelial-Mesenchymal Transition (EMT)

For TGF-β induced epithelial mesenchymal transition (EMT) images, HBE cells (50,000 cells/well) were seeded in a 6-well culture plate. Next day, all treatments (except the medium control) were starved with replacement of growth factor-absent media. After 24 hours incubation, the QA treatments were administered: 0.5, 1, 2.5, and 5 μM. Each treatment also included 10 ng/mL of TGF-β. The remaining 2 wells were treated with regular media and TGF-β as the negative control and positive control, respectively. Individual images of the cells were taken with an EVOS-FL Cell imaging fluorescence microscope (Thermo Fisher Scientific, Waltham, MA, USA) after 24 and 48 hours.

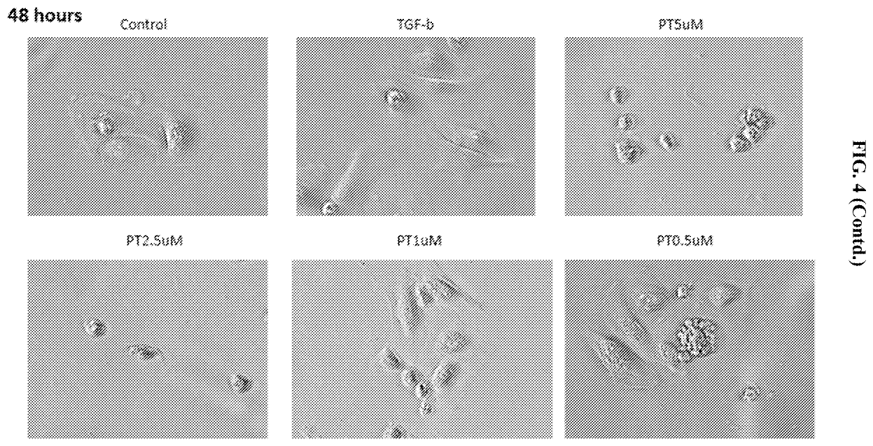

As shown in FIG. 4, QA treatment effectively inhibited TGF-beta induced EMT, at concentrations as low as 0.5-1 μM, at both 24- and 48-hour time-points. EMT is presented by morphological changes following TGF-beta treatment, and subsequent reversal with QA treatment.

These results demonstrate that compositions including quinacrine or pharmaceutically acceptable salts thereof are useful in methods for preventing, ameliorating or treating pulmonary fibrosis and/or reducing the severity of one or more risk factors, signs, or symptoms associated with pulmonary fibrosis.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method for treating or preventing idiopathic pulmonary fibrosis (IPF) in a subject in need thereof, comprising administering to the subject a therapeutically effective amount of quinacrine or a pharmaceutically acceptable salt thereof.

2. (canceled)

3. The method of claim 1, wherein the signs or symptoms of idiopathic pulmonary fibrosis comprise one or more of dry cough, chest pain, loss of appetite, shortness of breath, fatigue, weight loss, clubbing (a widening and rounding of the tips of the fingers), joint and muscle aches, swelling of the legs, epithelial to mesenchymal transition (EMT), myofibroblast activation, and severe progressive fibrosis including fibrotic foci and honeycombing.

4. The method of claim 3, wherein EMT is characterized by loss of epithelial markers, cytoskeletal reorganization, and transition to a spindle-shaped morphology with acquisition of mesenchymal markers.

5. The method of claim 1, wherein the subject is human.

6. The method of claim 1, wherein the quinacrine or pharmaceutically acceptable salt thereof is administered orally, topically, intranasally, systemically, intravenously, subcutaneously, intraperitoneally, intradermally, intraocularly, iontophoretically, transmucosally, or intramuscularly.

7. The method of claim 1, further comprising separately, sequentially or simultaneously administering one or more additional therapeutic agents to the subject.

8. The method of claim 7, wherein the additional therapeutic agents are selected from the group consisting of: nintedanib, pirfenidone, corticosteroids, and oxygen therapy.

9. The method of claim 1, wherein the subject exhibits reduced hyper-proliferation of lung fibroblasts, and/or decreased lung scarring following administration of quinacrine or pharmaceutically acceptable salt thereof.

10. The method of claim 1, wherein the subject exhibits a decrease in ECM production, reduced collagen expression, and/or an increase in E-cadherin following administration of quinacrine or pharmaceutically acceptable salt thereof.

11. The method of claim 1, wherein the quinacrine or pharmaceutically acceptable salt thereof is administered daily for 1 week or more.

12. The method of claim 1, wherein the quinacrine or pharmaceutically acceptable salt thereof is encapsulated in nanoparticles.

13. The method of claim 12, wherein the nanoparticles are PLGA hybrid nanoparticles.

14. The method of claim 1, wherein the IPF is induced by TGF-β signaling, PDGF-BB signaling, FGF signaling, or exposure to bleomycin.
